(19) United States
(12) Patent Application Publication
(10) Pub. No.: US 2026/0308459 A1
(43) Pub. Date: Oct. 8, 2026

(54) METHODS FOR HEAT INTEGRATION OF A HYDROCARBON UPGRADING SYSTEM INCLUDING AN ELECTRIFIED FURNACE

(71) Applicant: Dow Global Technologies LLC, Midland, MI (US)

(72) Inventors: Matthew Christopher Simons, Lake Jackson, TX (US); Georgios Bellos, Terneuzen (NL); Wim M. Kamperman, Middelburg (NL); Ryan C. Johnson, Rancho Cucamonga, CA (US); Richard Donaldson Cooper, Leiden (NL); Govert Gerardus Pieter van der Ploeg, Woerden (NL)

(73) Assignee: Dow Global Technologies LLC, Midland, MI (US)

(21) Appl. No.: 19/481,346

(22) PCT Filed: May 8, 2023
(86) PCT No.: PCT/GR2023/000018

§ 371 Date: Nov. 4, 2025

(51) Int. Cl. C10G 9/36; C07C 4/04

(52) U.S. Cl. CPC C10G 9/36; C07C 4/04; C10G 2300/1059; C10G 2300/1081

(57) ABSTRACT

A method of upgrading a feed stream may include introducing dilution steam to a feed stream, thereby forming a feed mixture, preheating the feed mixture, upgrading the feed mixture in an electrified hydrocarbon upgrading furnace including one or more electrical heating elements to form a cracked gas effluent, introducing the cracked gas effluent to a first heat exchanger thereby cooling the cracked gas effluent to a temperature in the range of 500° C. to 700° C., and further treating the cracked gas effluent to form a cracked gas. Wherein, a steam drum is fluidly connected to the first heat exchanger for transferring steam condensate to the first heat exchanger and saturated steam back to the steam drum, thereby forming a closed heat exchange loop, and the steam condensate transferred to the third heat exchanger is at a pressure in the range of 1 bar to 50 bar.

### FIELD

Embodiments of the present disclosure generally relate to the field of refining and upgrading hydrocarbons, and pertain particularly to a method of upgrading hydrocarbons using a heat integrated system and electrified furnace.

### BACKGROUND

Ethylene is widely used as an intermediate in the petrochemical industry and its production exceeds that of any other organic compound. Much of ethylene production goes to the manufacture of ethylene oxide, ethylene dichloride and polyethylene, which are precursors to a multitude of everyday consumer products. Despite various improvements over the years in thermal efficiency, reliability and safety, steam cracking furnaces used to form hydrocarbons such as ethylene remain heavily reliant on combustion of fossil fuels to provide process heat leading to substantial greenhouse gas emissions.

The steam cracking process to produce ethylene requires roughly half the energy required of competing processes (e.g., direct C) conversion technologies) and is projected to remain as the most energy efficient process. CO₂ emissions from steam cracking of ethane can reach the level of 1 ton-CO₂ per ton-ethylene produced and is lower than steam cracking of naphtha and alternates C₁-based routes to ethylene. At projected rates of ethylene production, CO₂ emissions from conventional steam cracking could exceed 300 Mta-CO₂ in the coming years. About 85% of the CO₂ emissions in the steam cracking process are emitted in the radiant combustion furnace section. In conventional radiant furnaces, numerous fuel gas (methane) burners are deployed to efficiently radiate heat from the combustion process through tubular reactor walls containing flowing feedstocks (e.g., hydrocarbon and steam) and product gases, providing heat to perform the required endothermic chemical reactions.

### SUMMARY

Accordingly, the growth and availability of renewable electricity creates an opportunity to use renewable energy in the formation of ethylene, eliminating the need to burn fossil fuels, and achieve a lower emission process. Various electric heating technologies such as impedance, induction, plasma, and microwaves may be used in place of combustion fired heating to generate and effectively transfer heat into the radiant coils of steam cracking furnaces. However, needs still exist for systems that can form ethylene and other high value chemicals from hydrocarbon feedstocks, such as naphtha, via heating with renewable electric sources.

However, in the application of electrical heating to large scale steam cracking processes, large amounts of electrical current and power are required. A key challenge is how to apply electrical heating efficiently and strategically to a large scale steam cracker via multiple conduits requiring many hundreds of Megawatts of electrical power. Reducing the required electrical power results in increased efficiency for the process and associated electrical connections and supporting equipment. Further, facilitating a tailored heat injection rate to the electrically conductive tubes increases desirable product yields.

Further reductions to the total required power input may be possible by effective heat integration of the system. Heat integration of the system may occur by capturing and utilizing all available heat within the process to effect the creation of the desired products. This should ideally be done within other process constraints, notably the minimization of fouling and side-reactions that have a deleterious effect on the total yield of desired products.

Accordingly, improved methods of upgrading hydrocarbons that minimize greenhouse gas emissions in a heat integrated cycle are desired. Systems and methods herein accomplish the aforementioned goals by the utilization of an electrified hydrocarbon upgrading furnace and heat integration through recycling of steam.

For example, and in embodiments described herein, steam may be used to effect heat transfer from the hotter steam to the cooler feed through heat exchangers within various locations of the system. The use of steam may reduce the electrical input required by the electrified furnace to initially heat and then upgrade the feed stream. Heat integration of the system may occur by recycling the condensed steam back to the steam source, wherein it may be used again to effect heat transfer in the system as an integrated cycle, thereby using utilizing all available heat within the method. Further yet gains may be realized by interacting the condensed steam with the output side of the electrified furnace. In steam cracking, the upgraded hydrocarbons exit the furnace at a greater temperature than at which they entered, even when powered by electric heating technology rather than conventional gas-fired heaters. The condensed steam may interact with the upgraded hydrocarbons through another heat exchanger, thereby heating the condensed steam, (which may also be referred to as “boiler-feed water”) and cooling the upgraded hydrocarbons. The now re-heated steam may then be recycled to the steam source, where it may be used in the integrated cycle. Thereby, all available heat within the system may be put towards upgrading hydrocarbons and a minimal amount of heat and energy lost or unused.

Moreover, the electrification of a process plant minimizes the use of steam as an energy source for driving machinery such as pumps, refrigerators. The conversion of steam enthalpy to shaft work may lead to 30-35% energy recovery, and the implementation of electric-drives can improve efficiency. Therefore reducing the export of steam from an ethylene furnace may be beneficial and/or desired. The use of lower pressure steam than typical with the output side of the electrified furnace may realize even greater gains due to the lower temperature and higher latent heat. Particularly, lower pressure steam may result in a faster quenching of the furnace effluent for a given length, size, or flow rate of a heat exchanger, reducing the amount of undesired side reactions that may affect the desired yield of the process as a whole. Another advantage of designing the system for lower pressure steam may be a more optimized preheat of liquid feeds.

Preheating of liquid feeds should ideally minimize the presence of the liquid fraction in contact with the hot tubes of the preheat heat exchangers. The remaining liquid film is exposed for a long time on a hot tube metal temperature which may lead to thermal reactions and therefore fouling. This may be traditionally observed in gas-fired furnaces wherein the heat is transferred from a hot flue gas stream to the feed stream. Accordingly, a lower pressure for the steam may result in cooler tubes, minimizing the extent of fouling thermal reactions within the preheat heat exchangers.

According to one embodiment, a method of upgrading a feed stream may include introducing dilution steam to the feed stream, thereby forming a feed mixture, preheating the feed mixture, upgrading the feed mixture in an electrified hydrocarbon upgrading furnace including one or more electrical heating elements to form a cracked gas effluent, introducing the cracked gas effluent to a first heat exchanger thereby cooling the cracked gas effluent to a temperature in the range of 500° C. to 700° C., and further treating the cracked gas effluent to form a cracked gas; wherein, a steam drum is fluidly connected to the first heat exchanger for transferring steam condensate to the first heat exchanger and saturated steam back to the steam drum, thereby forming a closed heat exchange loop, and the steam condensate transferred to the first heat exchanger is at a pressure in the range of 1 bar to 50 bar.

According to another embodiment, a method of upgrading a feed stream may include introducing the feed stream to a second heat exchanger, thereby preheating the feed stream to a temperature in the range of 23° C. to 263° C., introducing dilution steam to the feed stream, thereby forming a feed mixture, preheating the feed mixture, upgrading the feed mixture in an electrified hydrocarbon upgrading furnace including one or more electrical heating elements to form a cracked gas effluent, introducing the cracked gas effluent to a first heat exchanger thereby cooling the cracked gas effluent to a temperature in the range of 500° C. to 700° C., and further treating the cracked gas effluent to form a cracked gas; wherein, a steam drum is fluidly connected to the initial heat exchanger for transferring steam to the initial heat exchanger and steam condensate back to the steam drum, as well as fluidly connected to the first heat exchanger for transferring steam condensate to the first heat exchanger and saturated steam back to the steam drum, thereby forming a closed heat exchange loop, and the steam condensate transferred to the first heat exchanger is at a pressure in the range of 1 bar to 50 bar.

Additional features and advantages of the embodiments described herein will be set forth in the detailed description which follows, and in part will be readily apparent to those skilled in the art from that description or recognized by practicing the embodiments described, including the detailed description and the claims which are provided infra.

### BRIEF DESCRIPTION OF THE DRAWINGS

The following detailed description of specific embodiments of the present disclosure can be best understood when read in conjunction with the following drawings in which:

FIG. 1 illustrates a process flow diagram for an exemplary process in accordance with embodiments described herein;

FIG. 2 illustrates another process flow diagram for an exemplary process in accordance with embodiments described herein;

FIG. 3 illustrates another process flow diagram for an exemplary process in accordance with embodiments described herein;

FIG. 4 illustrates another process flow diagram for an exemplary process in accordance with embodiments described herein; and

FIG. 5 illustrates the relationship between the heat of vaporization vs. pressure for steam.

### DETAILED DESCRIPTION

Embodiments described herein relate to methods of upgrading a feed stream, such as a hydrocarbon feed stream including ethane or naphtha.

For the purpose of describing the simplified schematic illustrations and descriptions of the relevant figures, the numerous valves, temperature sensors, electronic controllers and the like that may be employed and well known to those of ordinary skill in the art of certain chemical processing operations are not included. Further, accompanying components that are often included in typical chemical processing operations, such as air supplies, and catalyst hoppers, are not depicted. Accompanying components that are in typical upgrading units, such as bleed streams, spent catalyst discharge subsystems, and catalyst replacement sub-systems are also not shown. It should be understood that these components are within the spirit and scope of the present embodiments disclosed. However, operational components, such as those described in the present disclosure, may be added to the embodiments described in this disclosure.

It should further be noted that arrows in the drawings refer to process streams. However, the arrows may equivalently refer to transfer lines, which may serve to transfer process streams between two or more system components. Additionally, arrows that connect to system components define inlets or outlets in each given system component. The arrow direction corresponds generally with the major direction of movement of the materials of the stream contained within the physical transfer line signified by the arrow. Furthermore, arrows, which do not connect two or more system components, signify a product stream, which exits the depicted system, or a system inlet stream, which enters the depicted system. Product streams may be further processed in accompanying chemical processing systems or may be commercialized as end products. System inlet streams may be streams transferred from accompanying chemical processing systems or may be non-processed feedstock streams. Some arrows may represent recycle streams, which are effluent streams of system components that are recycled back into the system. However, it should be understood that any represented recycle stream, in some embodiments, may be replaced by a system inlet stream of the same material, and that a portion of a recycle stream may exit the system as a product.

Additionally, arrows in the drawings may schematically depict process steps of transporting a stream from one system component to another system component. For example, an arrow from one system component pointing to another system component may represent “passing” a system component effluent to another system component, which may include the contents of a process stream “exiting” or being “removed” from one system component and “introducing” the contents of that product stream to another system component.

It should be understood that according to the embodiments presented in the relevant figures, an arrow between two system components may signify that the stream is not processed between the two system components. In other embodiments, the stream signified by the arrow may have substantially the same composition throughout its transport between the two system components. Additionally, it should be understood that in embodiments, an arrow may represent that at least 75 wt. %, at least 90 wt. %, at least 95 wt. %, at least 99 wt. %, at least 99.9 wt. %, or even 100 wt. % of the stream is transported between the system components. As such, in embodiments, less than all of the stream signified by an arrow may be transported between the system components, such as if a slip stream is present.

It should be understood that two or more process streams are “mixed” or “combined” when two or more lines intersect in the schematic flow diagrams of the relevant figures. Mixing or combining may also include mixing by directly introducing both streams into a like reactor, separation unit, or other system component. For example, it should be understood that when two streams are depicted as being combined directly prior to entering a separation unit or reactor, that in embodiments the streams could equivalently be introduced into the separation unit or reactor and be mixed in the reactor. Alternatively, when two streams are depicted to independently enter a system component, they may in embodiments be mixed together before entering that system component.

However, it should also be understood that when two or more process streams enter a heat exchanger, the streams are not mixed. Instead, the streams may communicate in terms of heat transfer from one stream to the other while not directly contacting one another.

Ranges can be expressed herein as from “in the range of” one particular value to another particular value. When such a range is expressed, another embodiment includes from the one particular value and/or to the other particular value. It will be further understood that the endpoints of each of the ranges are significant both in relation to the other endpoint, and independently of the other endpoint.

Additional features and advantages of the described embodiments, some embodiments of which are illustrated in the accompanying drawings, will be set forth in the detailed description, which follows, and in part will be readily apparent to those skilled in the art from that description or recognized by practicing the described embodiments, including the detailed description, which follows, as well as the claims.

As used herein, references to pressure in “bar,” “pascals,” or any equivalent pressure units may be understood to be in absolute pressure units, rather than in gauge pressure units.

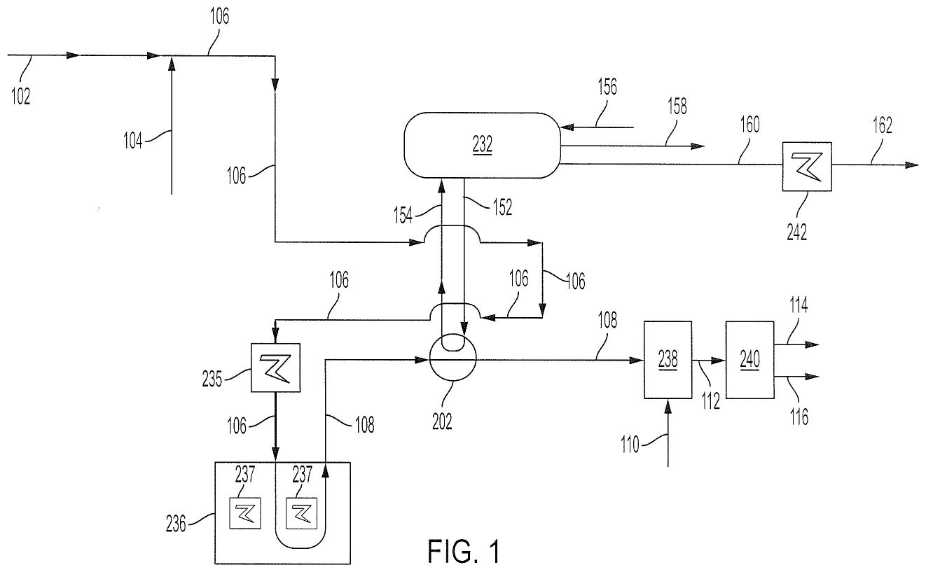

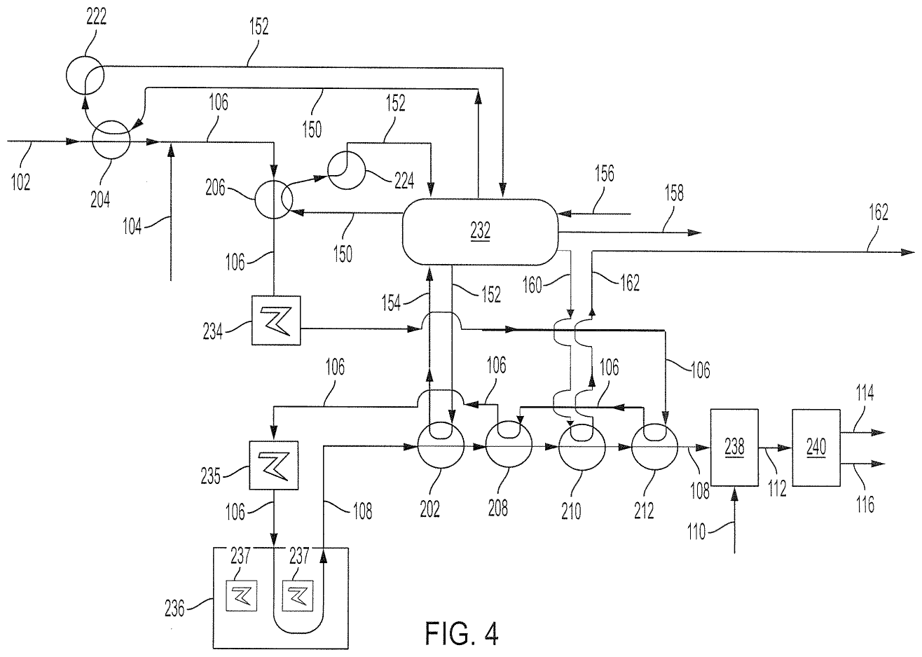

As previously stated, embodiments herein are directed to methods of upgrading a feed stream 102. The feed stream 102 may include hydrocarbon oils, refined hydrocarbons, bio-derived feeds, circular (recycled hydrocarbon) feeds, or combinations thereof. The hydrocarbon oil may be selected from the group consisting of naphthas, natural gas condensates, Fischer-Tropsch derivatives, gas-oils, pyrolysis oils, or combinations thereof. Additionally or alternatively, the feed stream 102 may include hydrocarbon gases selected from the group consisting of ethane, propane, butane, or combinations thereof. Now referring to FIGS. 1-4, the methods herein may include a system including an electrified hydrocarbon upgrading furnace 236; one or more heat exchangers 202, 204, 206, 208, 210, 212; one or more pumps 222, 224, one or more e-heaters 234, 235, 242;

or combinations thereof. The electrified hydrocarbon upgrading furnace 236, also referred to herein as “furnace” for short, may include one or more electrical heating elements 237 for upgrading a feed mixture 106. As used herein, “electrical heating elements” or “e-heater” may refer to one or more electrical heating technologies, including but not limited to direct electric heating, indirect electric heating, plasma, shockwave, microwave, or combinations thereof.

The method also includes introducing dilution steam 104 to the feed stream 102, thereby forming a feed mixture 106. The method may also include preheating the feed mixture. The method may also include upgrading the feed mixture 106 in an electrified hydrocarbon upgrading furnace 236 to form a cracked gas effluent 108. The cracked gas effluent 108 may then be introduced to a first heat exchanger 202, wherein the cracked gas effluent 108 may be cooled to a lower temperature than for the cracked gas effluent 108 within the electrified hydrocarbon upgrading furnace 236. The cracked gas effluent 108 may also be introduced to fourth 208, fifth 210, and sixth 212 heat exchangers to further cool the cracked gas effluent 108. The cracked gas effluent 108 may then be further treated to form a cracked gas 114. Without being limited by theory, the primary purpose of cooling the cracked gas effluent 108 may be to prevent further undesired reactions.

The method may also include an initial step of introducing the feed stream 102 to a second heat exchanger 204. Introducing the feed stream 102 to the second heat exchanger 204 may initially preheat the feed stream 102, which may be necessary when the feed stream 102 includes the hydrocarbon oils, the refined hydrocarbons, the bio-derived feeds, or combinations thereof. Moreover, the initial preheating may not be necessary when the feed stream only includes the hydrocarbon gases, as explained in further detail hereinbelow. Introducing the feed stream 102 to the second heat exchanger 204 may operate to vaporize at least a portion of the feed stream 102. Subsequently introducing the feed stream 102 to the dilution steam 104 may operate to vaporize the remaining portion of the feed stream 102, as the temperature of the feed mixture 106 may be above the dew point of the feed mixture 106. As previously mentioned, this initial preheating step may not be necessary where the feed stream consists of the hydrocarbon gases, as the feed stream 102 in this case would already be vaporized. Introducing the feed stream 102 to the second heat exchanger may initially preheat the feed stream to a temperature in the range of 23° C. to 263° C., such as from 23° C. to 50° C., from 50° C. to 70° C., from 70° C. to 100° C., from 100° C. to 120° C., from 120° C. to 140° C., from 140° C. to 160° C., from 160° C. to 190° C., from 190° C. to 210° C., from 210° C. to 230° C., from 230° C. to 263° C., or any combination of ranges or smaller range therein. For example, in embodiments, the feed stream 102 may be preheated to from 23° C. to 263° C. for hydrocarbon gas feeds, and from 70° C. to 263° C. for hydrocarbon oils, refined hydrocarbons, bio-derived feeds, circular feeds, or combinations thereof.

As previously stated, the method may include preheating the feed mixture 106.

Preheating the feed mixture 106 may include introducing the feed mixture 106 to a third heat exchanger 206, thereby preheating the feed mixture 106 to a temperature in the range of 70° C. to 263° C., such as from 70° C. to 100° C., from 100° C. to 120° C., from 120° C. to 140° C., from 140° C. to 160° C., from 160° C. to 190° C., from 190° C. to 210° C., from 210° C. to 230° C., from 230° C. to 263° C., or any combination of ranges or smaller range therein. For example, in embodiments, the feed mixture 106 may be preheated to from 23° C. to 263° C. for hydrocarbon gas feeds, and from 70° C. to 263° C. for hydrocarbon oils, refined hydrocarbons, bio-derived feeds, circular feeds, or combinations thereof. Preheating the feed mixture 106 in the third heat exchanger 206 may be generally understood to preheat the feed mixture 106 to a greater temperature than that exiting the second heat exchanger 204.

Preheating the feed mixture 106 may also or alternatively include introducing the feed mixture 106 to a fourth heat exchanger 208. The fourth heat exchanger 208 may be positioned downstream of the first heat exchanger 202 on a cracked gas effluent 108 side. The fourth heat exchanger 208 may also be positioned between the third heat exchanger 206 and the electrified hydrocarbon upgrading furnace 236 on a feed mixture 106 side. The fourth heat exchanger 208 may preheat the feed mixture 106 to a temperature in the range of 380° C. to 650° C. Preheating the feed mixture 106 in the fourth heat exchanger 208 may be generally understood to preheat the feed mixture 106 to a greater temperature than that exiting the second heat exchanger 204, the third heat exchanger 206, or both.

The fourth heat exchanger 208 may also cool the cracked gas effluent 108 to a temperature of less than 700° C. to greater than or equal to 150° C., such as from less than 700° C. to greater than or equal to 150° C. for hydrocarbon gas feeds, or from less than 700° C. to greater than or equal to 300° C. for hydrocarbon oils, refined hydrocarbons, bio-derived feeds, circular feeds, or combinations thereof. Particularly, a temperature of the cracked gas effluent 108 exiting the cracked gas effluent 108 side of the fourth heat exchanger 208 may be less than a temperature of the cracked gas effluent 108 exiting the first heat exchanger 202. In other words, the fourth heat exchanger 208 may further cool the cracked gas effluent 108 exiting the first heat exchanger 202.

The temperature of the cracked gas effluent 108 exiting the cracked gas effluent 108 side of the fourth heat exchanger 208 may be generally chosen to be high enough so that substantial fouling due to condensation of heavy components in the cracked gas effluent does not occur. When cracking gaseous feedstocks such as ethane, the cracked gas effluent may be generally cooled to 150-250° C., such as less than or equal to 250° C., while liquid feedstocks such as naphtha may be generally cooled to temperatures greater than 300° C.

Preheating the feed mixture 106 may also include introducing the feed mixture 106 to a first e-heater 234, wherein the first e-heater 234 may be positioned between the third heat exchanger 206 and the feed mixture side 106 side of the fourth heat exchanger 208. The first e-heater 234 may operate to preheat the feed mixture 106 to a temperature of 250° C. to 400° C., such as to avoid fouling the cracked gas effluent 108 in the fourth heat exchanger 208, as explained in further detail hereinbelow.

Preheating the feed mixture 106 may also or alternatively include introducing the feed mixture 106 to a sixth heat exchanger 210. The sixth heat exchanger 212 may be positioned downstream of the third heat exchanger 206 and upstream of the fourth heat exchanger 208 on the feed mixture 106 side. The sixth heat exchanger 212 may also be positioned downstream of the fourth heat exchanger 208 on the cracked gas effluent 108 side. The sixth heat exchanger 210 may preheat the feed mixture 106 to a temperature in the range of 250° C. to 650° C., as well as cooling the cracked gas effluent 108 to a temperature of less than 700° C. to greater than or equal to 150° C., similar to the fourth heat exchanger 208, such as to greater than or equal to 150° C. or from 150° C. to 250° C. for hydrocarbon gas feeds. Similarly, for hydrocarbon oils, refined hydrocarbons, bio-derived feeds, circular feeds, or combinations thereof, the sixth heat exchanger 210 may cool the cracked gas effluent to 108 to a temperature of less than or equal to 350° C., such as from greater than or equal to 300° C. to less than or equal to 350° C.

It is contemplated that there may be some risk in using the cracked gas effluent 108 in the fourth and sixth heat exchangers 208, 212 alongside the feed mixture 106. For example, cooling the cracked gas effluent 108 on the ‘hot side’ (cracked gas effluent 108 side) of the fourth or sixth heat exchangers 208, 212 past the dew point of a significant fraction of heavy compounds in the cracked gas effluent 108 may result in excessive condensation of said heavy fractions of the cracked gas effluent 108 on the internal surfaces of the heat exchangers, otherwise known as “fouling.” However, in embodiments, this risk is minimized by the pre-heating of the feed mixture 106 to a temperature in the range of 250° C. to 400° C., such as by utilizing the first e-heater 234, before entry into the fourth or sixth heat exchangers 208, 212 on the ‘cold side’ (feed mixture 106 side).

Preheating the feed mixture 106 may also include introducing the feed mixture 106 to a second e-heater 235, wherein the second e-heater 235 may be positioned between the fourth heat exchanger 208 on the feed mixture 106 side and the electrified hydrocarbon upgrading furnace 236, such as immediately before (i.e. immediately upstream) the electrified hydrocarbon upgrading furnace 236. The second e-heater 235 may operate to preheat the feed mixture 106 to a temperature in the range of 120° C. to 500° C.

It is contemplated that the use of the second e-heater 235 may add flexibility to the methods herein in terms of possible feed streams 102. For example, and in embodiments, the temperature at which the feed mixture 106 is preheated to may depend on the composition of the feed stream 102. As the average carbon content of the feed mixture 106 decreases, the temperature to which the feed mixture 106 needs to be heated to upgrade the feed into the cracked gas effluent 108 may in turn increase. This additional heating burden may be borne by either the preheating methods or the electrified hydrocarbon upgrading furnace 236. However, the possible heating burden that may be borne by the furnace 236 may be limited by the dimensions of the furnace 236, such that progressive decreases in carbon content, such as swapping the feed mixture from primarily naphtha to primarily ethane, may require a larger furnace to replace the comparatively smaller furnace. The time and cost to swap furnaces may in turn result in considerable economic losses to the method as a whole. Further, the potential heating burden that may be borne by the heat exchangers 202 and 204 may be limited by the temperature to which the steam 150 may be heated.

Methods herein may include a heat transfer fluid, as well as a storage medium for a heat transfer fluid. The heat transfer fluid may include steam 150. The storage medium may include a steam drum 232 for the steam 150. The steam drum 232 may be fluidly connected to the first and second heat exchangers 202 and 204 for transferring the steam 150 to the first and second heat exchangers 202 and 204 and steam condensate 152 back to the steam drum 232. The steam drum 232 may also be fluidly connected to the first heat exchanger 202 for transferring the steam condensate 152 to the first heat exchanger 202 and saturated steam 154 back to the steam drum 232. Introducing the steam condensate 152 to the first heat exchanger 202 may heat the steam condensate 152, thereby forming the saturated steam 154 (saturated mixture of steam and water).

The connection of the steam drum 232 to each of the heat exchangers 202, 204, 206 may thereby formed a “closed heat exchange loop.” As used herein, a “closed heat exchange loop” may not be entirely self-contained. For example, and in embodiments, the steam drum 232 may further include a purge valve 158, which may also be commonly referred to as a “blowdown valve.” As will be understood in the art, the purge valve 158 may operate to intentionally waste or ‘bleed’ water and steam from the steam drum 232 through the purge valve 158 to avoid concentration of impurities during continued use and evaporation of the steam 150. The steam drum may also include a feed valve 156. The feed valve 156 may operate to introduce water into the steam drum 232 to replace the steam and water bled from the purge valve 158.

As used herein, “film boiling” generally refers to a phenomenon that occurs when heat flux from a hot to cold stream across the wall of a heat exchanger causes a film of remaining liquid of the fluid being heated to form and at least partially cover the heating surface. This film of liquid becomes trapped between the majority of the fluid and the heating surface, and thereby significantly increases the residence time of the boiling liquid leading to initiating thermal reactions which cause fouling”. For example herein, film boiling may cause fouling of the feed stream 102 to form within the heat exchangers, thereby reducing the effectiveness of the heat exchangers, and in severe instances, critical failure of the materials of the heat exchanger.

In embodiments, the method may also include exporting the excess steam 160 from the steam drum 232, not otherwise sent to 204 or 206, from the cracker furnace section to another part of the plant/facility for use as process heating applications. Moreover, the excess steam 160 may be sent first to a third e-heater 242. Introducing the excess steam 160 to the third e-heater 242 may operate to form a superheated steam 162. The superheated steam 162 may be exported for use in any one of a number of further downstream units, including for process heating applications. Electrification of the cracker furnace 237 and the use of lower pressure steam to help integrate the heat between the feed and effluent disclosed herein, dramatically reducing the amount of thermodynamic work, such that all major steam turbines in the downstream section of the cracker plant may be electrified. The third e-heater 242 may heat the excess steam 160 from the steam drum 232 to a temperature in the range of 120° C. to 500° C. It is contemplated that one use for exported steam 160 may be in heating and/or vaporizing the dilution steam 104.

In additional embodiments, the system may include the fifth heat exchanger 210. The method may include introducing the excess steam 160 from the steam drum 232 to the fifth heat exchanger 210. The fifth heat exchanger 210 may be positioned downstream of the first and fourth heat exchangers on a cracked gas effluent 108 side. The fifth heat exchanger 210 may also contribute to the heating duty of the third e-heater 242 in producing the superheated stream 162. Particularly, the fifth heat exchanger 210 may heat the excess steam 160 from the steam drum 232 to a temperature in the range of 120° C. to 500° C., thereby producing the superheated steam 162. Further, the fifth heat exchanger 210 may further cool the cracked gas effluent 108 to a temperature of less than 700° C. to greater than or equal to 150° C., such as from less than 700° C. to greater than or equal to 150° C. for hydrocarbon gas feeds, or from less than 700° C. to greater than or equal to 350° C. for hydrocarbon oils, refined hydrocarbons, bio-derived feeds, circular feeds, or combinations thereof. Particularly, a temperature of the cracked gas effluent 108 exiting the cracked gas effluent 108 side of the fifth heat exchanger 208 may be less than a temperature of the cracked gas effluent 108 exiting the first heat exchanger 202, the third heat exchanger 208, or both. In other words, the fifth heat exchanger 210 may further cool the cracked gas effluent 108 exiting the first heat exchanger 202, the third heat exchanger 208, or both.

As previously noted, methods herein may include one or more pumps 222, 224. For example, and in embodiments, each of the second through third heat exchangers 202, 204, may further include pumps, or the pumps may be a separate unit. For example, and in embodiments, the pumps 222 and 224 may be interposed between the downstream end of the second and third heat exchangers 202 and 204, and the steam drum 232. The pumps 222 and 224 may operate to convey the steam condensate 152 exiting the second and third heat exchangers 202 and 204 back to the steam drum 232, wherein the steam condensate 152 may be re-heated for re-use as the steam 150, such as by transfer to the first exchanger 202 for cooling the cracked gas effluent 108.

As previously stated, introducing the steam condensate 152 to the first heat exchanger 202 may heat the steam condensate 152, thereby forming the saturated steam 154. However, the introduction of the steam condensate 152 to the first heat exchanger 202 may also operate to cool the cracked gas effluent 108, as previously described. Without being limited by theory, the cracked gas effluent 108 may undergo further reactions at its elevated temperature exiting the electrified hydrocarbon upgrading furnace 236. These further reactions may form undesired side-products, such that the cracked gas effluent is desired to be cooled in as quickly of a manner as possible to preserve the desired products. Without being limited by theory, a majority of these side reactions may be suppressed at temperatures below approximately 600-650° C., although at least a portion of the side-reactions may be suppressed at temperatures between 650° C. and 700° C. Accordingly, introducing the steam condensate 152 to cool the cracked gas effluent 108 may quench and/or suppress at least a portion of the side-reactions of the cracked gas effluent 108.

In embodiments, the steam 150, steam condensate 152, or excess steam 160 introduced to the heat exchangers 202, 204, 206, and 210 may be at a pressure in the range of 1 bar to 50 bar, such as from 1 bar to 5 bar, from 5 bar to 10 bar, from 10 bar to 15 bar, from 15 bar to 25 bar, from 25 bar to 30 bar, from 32 bar to 35 bar, from 35 bar to 38 bar, from 38 bar to 40 bar, from 40 bar to 50 bar, or any combination of ranges or smaller range therein, such as from 1 bar to 40 bar, such that the steam condensate 52 may be considered “lower pressure steam.” Particularly the pressure range may preferably be from 10 bar to 35 bar or from 10 bar to 15 bar for hydrocarbon gas feed streams and from 32 to 38 bar for hydrocarbon oil feed stream. Without being limited by theory, the steam used within hydrocarbon upgrading processes is ordinarily “high pressure steam,” in the range of 80-180 bar, and most commonly 100-120 bar. This may be primarily due to the high pressure steam ordinarily being exported to downstream steam turbines to make thermodynamic work to run various large compressor units and steam turbines in downstream processes. However, when exported steam is not otherwise desired, such as in an overall electrified cracker process as contemplated herein, the steam condensate 152 used in the first heat exchanger 202, as well as the steam 150 used in heat exchangers 204 and 206, and the excess steam 160 used in heat exchanger 210, may be lower pressure steam. The lower pressure steam used in steam condensate 152, steam 150, or excess steam 160 may have numerous benefits to the process, as detailed hereinbelow and in the examples section.

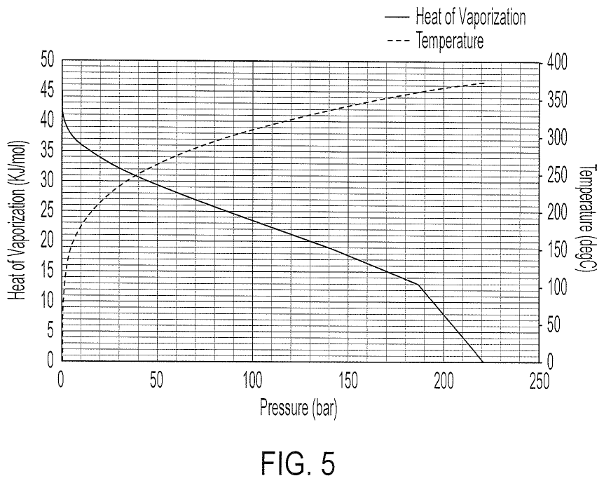

For example, the lower pressure steam may have a higher heat of vaporization than the relatively greater pressure steam. “heat of vaporization,” also referred to as the “enthalpy of vaporization,” is generally understood as the amount of heat need to turn 1 gram of a liquid (steam condensate in this case) into a vapor, without a rise in the temperature of the liquid. This is illustrated for example in FIG. 5, which shows the heat of vaporization of steam as a function of the steam's pressure. As shown in FIG. 5, as the pressure of the steam increases, the heat of vaporization decreases. In contrast, as the pressure of the steam decreases, the heat of vaporization increases. Accordingly, it is contemplated that lower pressure steam can more effectively transfer heat for a given exposure length of a heat exchanger, or for a given flow rate within the same.

Without being limited by theory, this may apply both when steam condensate 152 is partially vaporized in heat exchanger 202 while cooling the cracked gas effluent 108, as well as when steam 150 is condensed to preheat the feed 102 in the second or third heat exchangers 204 and 206. Stated in another way, the lower pressure steam may be considered as a more dense energy carrier to effect heat transfer. Further, lower pressure steam may have a lower temperature at saturation compared to higher pressure steam, as also shown in FIG. 5. The lower temperature of the condensate and steam at saturation may further increases the rate of heat transfer in 202 when used to cool the cracked gas effluent 108, such as by increasing the temperature gap between the hot and cold sides of the heat exchanger. The net effect may be a compounding of the advantages of the higher heat of vaporization and the lower temperature at saturation, thus contributing to unexpected gains in quenching efficiency and in suppressing the occurrence of side reactions.

Finally, the lower pressure and thus temperature of steam 150 reduces the temperature gradient across the heat exchangers 204/206 when it is condensed to form steam condensate 152. This reduced temperature gradient may decrease the likelihood of film boiling in 204/206, as well as suppressing the occurrence of side reactions of the feed stream 102 and the feed mixture 106.

As shown in the Examples hereinbelow, the use of 1 bar to 50 bar steam condensate 152, and preferably 10 bar to 40 bar steam in the first heat exchanger 202 may allow the cracked gas effluent 108 to be cooled to a temperature in the range of 500° C. to 700 with a shorter residence time for equivalent heat exchangers than for 100 bar to 120 bar steam condensate 152. In embodiments, any remainder of the cooling of the cracked gas effluent 108 necessary to recover heat and further suppress the remaining side-reactions may then be accomplished by introduction of the cracked gas effluent 108 into the fourth heat exchanger 208, as in embodiments herein. The net result may be an increase in the amount of desired products in the cracked gas effluent 108 with a corresponding minimization in the amount of side reactions producing undesired products in the same.

As previously stated, the cracked gas effluent 108 may be further treated to form a cracked gas 114. The further treatment may include exposing the cracked gas effluent 108 to a quench fluid 110 in a quench tower 238, thereby forming a quenched cracked gas mixture 112. In embodiments including the hydrocarbon gases, the quench fluid 110 may include water. Alternatively, in embodiments including the hydrocarbon oil, refined hydrocarbons, or bio-derived feeds, the quench fluid 110 may include diesel oil, gas oil, or both. The further treatment may also include introducing the quenched cracked gas mixture 112 to a separator 240, thereby forming a recovered fluid 116 and a cracked gas 114. The recovered fluid 116 may include tar in the case of diesel oil and/or gas oil quench fluids 110, and additional water in the case of water quench fluids 110. The cracked gas 114 may include olefins, as well as other hydrocarbon fractions. The quench tower 238 may be any quench tower generally understood in the art. The recovered fluid 116 may be further treated to recycle the quench fluid 110 for reuse in the quench tower 238, including by any method generally understood in the art.

In embodiments, and as previously stated, the methods herein may operate to reduce the required energy input/electrical power to upgrade a feed stream 102 by the use of an electrified hydrocarbon upgrading furnace and heat integration of the method. Methods herein may reduce the energy input over conventional methods including a convection based hydrocarbon upgrading furnace by at least 35%. Methods herein may also reduce the energy input over comparative methods with no heat integration loops by at least 10%. This is further shown and illustrated in the Examples hereinbelow.

### EXAMPLES

Simulations of the feed stream upgrading process, according to the embodiments herein, were performed using ASPEN PLUS® V11 software, utilizing APV110 Ethylene and APV110 PURE physical property databases with Soave-Redlich-Kwong equations of state.

### Example 1—Naphtha Cracker

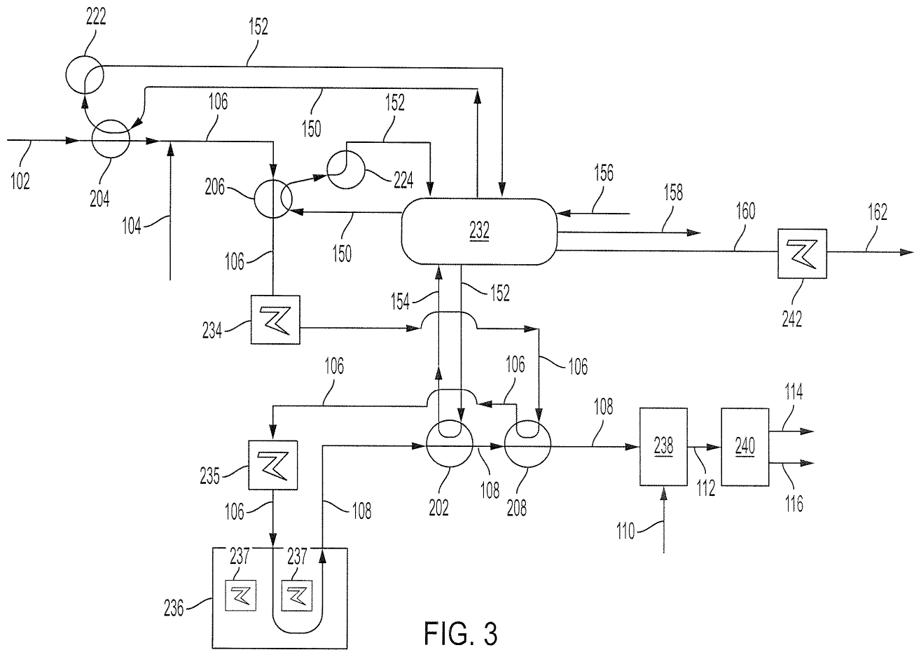

In Example 1, a 20,000 kg per hour naphtha stream (as the feed stream 102) was upgraded according to the embodiment illustrated in FIG. 3. The naphtha was first preheated to a temperature of 120° C. in the second heat exchanger 204 by condensing steam to provide the heat to vaporize at least a portion of the naphtha. The naphtha was then mixed with 10,000 kg per hour dilution steam 104 at 180° C. and 7 bar to form the feed mixture 106 at 125° C., and as previously discussed, vaporize the remaining portion of the naphtha. The feed mixture 106 was then introduced to the third heat exchanger 206 to preheat the feed mixture to a temperature of approximately 212° C. The feed mixture 106 was then preheated to a temperature of 300° C. and 535.7° C. in the first e-heater 234 and the fourth heat exchanger 208 respectively.

The second e-heater 235 further preheats the feed mixture 106 to 600° C. before upgrading in the electrified hydrocarbon upgrading furnace 236. The cracked gas effluent exiting the electrified hydrocarbon upgrading furnace 236 was at 847° C. and approximately 2.1 bar. The cracked gas effluent 108 was then rapidly cooled to 600° C. in the third heat exchanger by partially vaporizing 36 bar steam condensate 152. Particularly, the steam condensate 152 absorbed the excess heat from the cracked gas effluent 108 so that a fraction of the condensate forms saturated steam mixture 154. The cracked gas effluent was then further cooled to 360° C. in the fourth heat exchanger 208. The cracked gas effluent 108 was then further treated to form the cracked gas 114, according to FIG. 3.

The results of this method were then compared to a similar system (Comparative Design 1) utilizing a combustion-based hydrocarbon upgrading furnace, wherein high-pressure steam is formed by cooling the cracked gas in one or more transfer line exchanger using high-pressure steam condensate, and superheated by the flue gas from the combustion furnace, which also preheats the feed. The superheated high-pressure steam is exported in Comparative Design 1, and used to generate mechanical work in the downstream section of the plant. Identical mass flow rates, temperature, and pressure of the feed, dilution steam, and effluent leaving the furnace were assumed, i.e. that the performance of the conventional and electric furnaces were that same in reacting the hydrocarbon feed. The mass flow balance as well as the power usage in the comparison is shown below in Table 1. For the conventional furnace, the firebox efficiency is 42% and overall efficiency of 94%.

Naphtha feed composition and cracking yields were at least partially based on those from S. M. Sadrameli & A. E. S. Green, Systematics and modeling representations of naphtha thermal cracking for olefin production, Journal of Analytical and Applied Pyrolysis, 305-313 (Volume 73, Issue 2, 2005). Estimation of heat of reaction was calculated by comparing the cracked gas product composition with the initial naphtha composition, utilizing ASPEN PLUS® v11 software.

TABLE 1
Mass Flow Balance and Duty Comparison

| | | |
|---|---|---|
| | Comparative | Example 1 |
| Units | Design 1 | Design |

Mass Flow Feed Rates

| | | | |
|---|---|---|---|
| Naphtha Feed (60° C., 6 bar) | kg/h | 20,000 | 20,000 |
| Dilution Steam (180° C., 7 bar) | kg/h | 10,000 | 10,000 |
| Boiler Feed Water (120° C., 130 | kg/h | 25,117 | 0 |
| bar) | | | |
| Boiler Feed Water (120° C., 36 | kg/h | 0 | 4083 |
| bar) | | | |

Exported Saturated and Superheated Steam

| | | | |
|---|---|---|---|
| Steam Flow Rate (500° C., 110 | kg/h | 24,683 | 0 |
| bar) | | | |
| Steam Flow Rate (242° C., 36 | kg/h | 0 | 3841 |
| bar) | | | |
| Turbine Shaft Work Produced | kW | 7307 | 0 |

Duty Comparison

| | | | |
|---|---|---|---|
| Methane (20° C., 1 bar) | kg/h | 2915 | 0 |
| Combustion Air (20° C., 1 bar) | kg/h | 55,713 | 0 |
| Flue Gas (154° C., 1 bar) | kg/h | 58,627 | 0 |
| CO₂ Emissions | kg/h | 7997 | 0 |
| Electric Furnace Duty | kW | 0 | 17016 |
| Electric Heater Duty | kW | 0 | 3438 |
| Make-up Shaft Work | kW | 0 | 7307 |
| Total Furnace Duty | kW | 40,511 | 28,968 |

As shown in Table 1, the Comparative Design 1 produced about 7307 kW of excess Shaft Work in the combustion based furnace, which may be ordinarily exported to downstream cracker processes for use. The Example 1 in contrast did not produce any excess Shaft Work due to the electrified design and lower pressure steam. Accordingly, for a true comparison of total duty for the Examples an additional 7307 KW of make-up shaft work was produced for the electrified furnace. Accordingly, the electrified heating furnace required much less net energy (28,968 kW to 40,511 kW) than the equivalent system using the combustion-based hydrocarbon upgrading furnace. Moreover, the lower pressure (36 bar) steam design also resulted in much less boiler feedwater stream needing to be fed to the steam drum to keep the method operational. The lower pressure (36 bar) steam design also resulted in an improved initial quenching of the cracked gas effluent 108 (faster quench) as shown below in Table 2.

Particularly, for the lower pressure steam design, less residence time, length of the heat exchanger, and steam condensate flow rate were required to cool the cracked gas effluent to 600° C. than an equivalent higher pressure (110 bar) steam design. As previously discussed, a faster quench may operate to prevent undesired side reactions in the cracked gas effluent, which may improve yield of desired products (ethylene, propylene, butylene, etc.) For the design of the heat exchangers, ASPEN Exchanger Design and Rating (EDR) v11 software was used, with a single double-pipe exchanger (Tube outer diameter 96 mm, thickness 8 mm) to cool 1200 kg/h of cracked gas effluent against saturated liquid water with a vapor quality of 10% of the steam mixture leaving the heat exchanger.

TABLE 2
Quenching Comparison of Low and High Pressure
Steam Designs for Example 1 Naphtha Cracker

| | | | | Heat |
|---|---|---|---|---|
| Steam | Steam | | | Exchanger |
| Condensate | Condensate | Steam | Heat | Average |
| Pressure in | Temperature | Condensate | Exchanger | Residence |
| 3rd TLHE | in 3rd TLHE | Flow Rate | Length | Time |
| Bar | deg C. | kg/h | meters | seconds |
| 110 | 309 | 7536 | 6.59 | 0.058 |
| 36 | 242 | 5404 | 5.55 | 0.049 |

The composition of the naphtha feed and the resulting composition of the cracked gas effluent after cooling according to Example 1 is shown below in Table 3.

TABLE 3
Feed and Effluent Compositions

| | Weight Fraction | | | Weight Fraction | |
|---|---|---|---|---|---|
| | | Cracked | | | Cracked |
| | Naphtha | Gas | | Naphtha | Gas |
| Compound | Feed | Effluent | Compound | Feed | Effluent |
| HYDROGEN | 0 | 0.0108 | METHYL- | 0 | 0.0095 |
| | | | ACETYLENE | | |
| METHANE | 0 | 0.1666 | 1-BUTENE | 0 | 0.0416 |
| ETHANE | 0 | 0.0641 | 1,3-BUTADIENE | 0 | 0.0456 |
| PROPANE | 0 | 0.005 | CYCLOPENTADIENE | 0 | 0.0325 |
| N-BUTANE | 0.0453 | 0.0025 | BENZENE | 0.0217 | 0.0592 |
| N-PENTANE | 0.2252 | 0 | TOLUENE | 0.0037 | 0.0272 |
| N-HEXANE | 0.1202 | 0 | O-XYLENE | 0.0044 | 0.0066 |
| N-HEPTANE | 0.0169 | 0 | ETHYLBENZENE | 0 | 0.0005 |
| N-OCTANE | 0.0063 | 0 | STYRENE | 0 | 0.0082 |
| N-NONANE | 0.0007 | 0 | ISOPROPYLBENZENE | 0 | 0.0048 |
| ISOBUTANE | 0.0012 | 0 | NAPHTHALENE | 0 | 0.0321 |
| 2-METHYL- | 0.1648 | 0 | CYCLOPENTANE | 0.0738 | 0 |
| BUTANE | | | | | |
| 2-METHYL- | 0.1217 | 0 | CYCLOHEXANE | 0.07112 | 0 |
| PENTANE | | | | | |
| 2- | 0.002 | 0 | CARBON-MONOXIDE | 0 | 0.003 |
| METHYLHEPTANE | | | | | |
| ETHYLENE | 0 | 0.3195 | CARBON-DIOXIDE | 0 | 0.0005 |
| ACETYLENE | 0 | 0.0063 | 2,2-DIMETHYL- | 0.003 | 0 |
| | | | BUTANE | | |
| PROPYLENE | 0 | 0.1437 | 2,3-DIMETHYL- | 0.0118 | 0 |
| | | | BUTANE | | |

### Example 2—Ethane Cracker

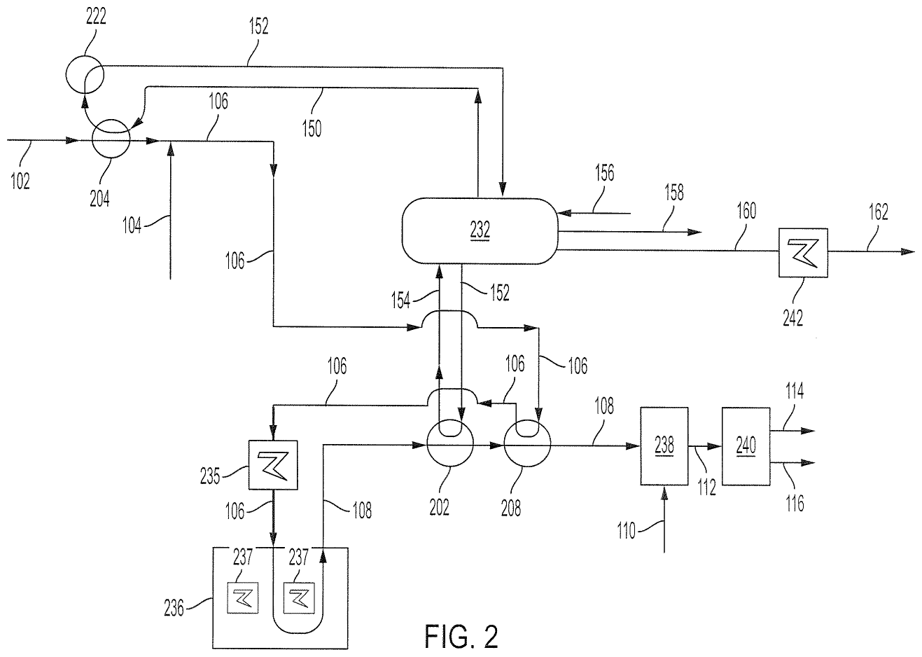

In Example 2, a 20,000 kg per hour ethane feed stream (35° C., 10 bar) was upgraded according to the embodiment illustrated in FIG. 2. Ethane was first preheated to a temperature of 163° C. in the second heat exchanger 204. The ethane was then mixed with 6,000 kg per hour dilution steam 104 at 180° C. and 7 bar to form the feed mixture 106 at 167° C. and 5.9 bar. The feed mixture 106 was then heated to a temperature of 620° C. in the fourth heat exchanger 208. The second e-heater 235 preheated the feed mixture to 690° C. before upgrading in the electrified hydrocarbon upgrading furnace 236.

The cracked gas effluent exiting the electrified hydrocarbon upgrading furnace 236 was at 850° C. and approximately 2.0 bar. The cracked gas effluent 108 was then rapidly cooled to 650° C. in the first heat exchanger with 13.5 bar steam condensate 152, to which the steam condensate 152 absorbs the heat from the cracked gas effluent 108 to form a mixture of condensate and saturated steam 154. The cracked gas effluent was then further cooled to 220° C. in the fourth heat exchanger 208. The cracked gas effluent 108 was then further treated to form the cracked gas 114, according to FIG. 2.

The results of this method were then compared to a similar system (Comparative Design 2) utilizing a combustion-based ethane upgrading furnace, wherein high-pressure steam is formed by cooling the cracked gas in one or more transfer line exchanger using high-pressure steam condensate, and superheated by the flue gas from the combustion furnace, which also preheats the feed. The superheated high-pressure steam is exported in Comparative Design 2, and used to generate mechanical work in the downstream section of the plant. Identical mass flow rates, temperature, and pressure of the feed, dilution steam, and effluent leaving the furnace were assumed, i.e. that the performance of the conventional and electric furnaces were the same in reacting the hydrocarbon feed. The mass flow balance as well as the power usage in the comparison is shown below in Table 4. For the conventional furnace, the firebox efficiency is 42% and overall efficiency of 93%.

Similarly to the naphtha cracking, ethane cracking yields were adapted from reported yields from Chapter: Ethylene (pg. 477, Table 3) by H. Zimmermann and R. Walzl in Ullman's Encyclopedia of Industrial Chemistry 2012 Wiley-VCH Verlag Gmbh & Co. KGaA, Weinheim and shown in Table 5. Estimation of the heat of reaction was calculated by comparing the cracked gas product composition with the initial ethane composition, utilizing ASPEN PLUS® v11 software.

TABLE 4
Mass Flow Balance and Duty Comparison

| | | |
|---|---|---|
| | Comparative | Example 2 |
| Units | Design 2 | Design |

Mass Flow Feed Rates

| | | | |
|---|---|---|---|
| Ethane Feed (35° C., 10 bar) | kg/h | 20,000 | 20,000 |
| Dilution Steam (180° C., 7 bar) | kg/h | 6,000 | 6,000 |
| Boil Feed Water (120° C., 130 | kg/h | 33,505 | 0 |
| bar) | | | |
| Boiler Feed Water (120° C., 15 | kg/h | 0 | 6,009 |
| bar) | | | |

Exported Saturated and Superheated Steam

| | | | |
|---|---|---|---|
| Steam Flow Rate (500° C., 108 | kg/h | 32,892 | |
| bar) | | | |
| Steam Flow Rate (194° C., 13.5 | kg/h | 0 | 5,831 |
| bar) | | | |
| Turbine Shaft Work Produced | kW | 9,749 | 0 |

Duty Comparison

| | | | |
|---|---|---|---|
| Methane (60° C., 1 bar) | kg/h | 3,496 | 0 |
| Combustion Air (25° C., 1 bar) | kg/h | 68,458 | 0 |
| Flue Gas (180° C., 1 bar) | kg/h | 71,954 | 0 |
| CO₂ Emissions | kg/h | 9,591 | 0 |
| Electric Furnace Duty | kW | 0 | 20,410 |
| Electric Heater Duty | kW | 0 | 1798 |
| Make-up Shaft Work | kW | 0 | 9,749 |
| Total Furnace Duty | kW | 48,486 | 32,575 |

As shown in Table 4, the Comparative Design 2 produced about 9,749 kW of excess Shaft Work in the combustion based furnace, which may be ordinarily exported to downstream refinery processes for use. The Example 2 in contrast did not produce any excess Shaft Work due to the electrified design. Accordingly, for a true comparison of total duty for the Examples an additional 9,749 kW of make-up shaft work was produced for the electrified furnace. Accordingly, the electrified heating furnace required much less net energy (32,575 kW to 48,486 kW) than the equivalent system using the combustion-based hydrocarbon upgrading furnace. Moreover, the lower pressure (13.5 bar) steam design also results in much less boiler feedwater stream needing to be fed to the steam drum to keep the method operational. The lower pressure (13.5 bar) steam design also resulted in an improved initial quenching of the cracked gas effluent 108, similar to Example 1. Particularly, for the lower pressure design, less residence time was required to cool the cracked gas effluent 108 to 650° C. than the higher pressure (110 bar) steam design.

TABLE 5
Feed and Effluent Compositions (dry basis)

| | Weight Fraction | | | Weight Fraction | |
|---|---|---|---|---|---|
| | | Cracked | | | Cracked |
| | Ethane | Gas | | Naphtha | Gas |
| Compound | Feed | Effluent | Compound | Feed | Effluent |
| Hydrogen | 0 | 0.0409 | Butadiene | 0 | 0.0179 |
| Methane | 0 | 0.0319 | 1-Butene | 0 | 0.0018 |
| Acetylene | 1 | 0.0054 | Butane | 0 | 0.0021 |
| Ethylene | 0 | 0.5285 | Benzene | 0 | 0.0038 |
| Ethane | 0 | 0.3499 | Toluene | 0 | 0.0006 |
| Methyl-acetylene | 0 | 0.0002 | Styrene | 0 | 0.0002 |
| Propylene | 0 | 0.0106 | Naphthalene | 0 | 0.0040 |
| Propane | 0 | 0.0012 | Carbon | 0 | 0.0003 |
| | | | Monoxide | | |
| Vinyl-acetylene | 0 | 0.0005 | Carbon | 0 | 0.0001 |
| | | | Dioxide | | |

Assumed in the previous simulations (Examples 1 and 2) was a heat loss of 2% in all heat exchangers. Shaft Work was calculated by passing steam 154 through a turbine with isentropic efficiency 0.8, mechanical efficiency 1, to 2 in-Hg condensate effluent pressure. Steam drum blow down calculated as 2% of the condensate and fresh boiler feed water to the steam drum. Flowrate of the steam condensate was set at that which would achieve 10% vapor quality at the outlet of the heat exchanger 202.

It is noted that recitations in the present disclosure of a component of the present disclosure being “operable” or “sufficient” in a particular way, to embody a particular property, or to function in a particular manner, are structural recitations, as opposed to recitations of intended use. More specifically, the references in the present disclosure to the manner in which a component is “operable” or “sufficient” denotes an existing physical condition of the component and, as such, is to be taken as a definite recitation of the structural characteristics of the component.

The singular forms “a,” “an” and “the” include plural referents, unless the context clearly dictates otherwise.

Throughout this disclosure ranges are provided. It is envisioned that each discrete value encompassed by the ranges are also included. Additionally, the ranges which may be formed by each discrete value encompassed by the explicitly disclosed ranges are equally envisioned.

As used in this disclosure and in the appended claims, the words “comprise,” “has,” and “include” and all grammatical variations thereof are each intended to have an open, non-limiting meaning that does not exclude additional elements or steps.

As used in this disclosure, terms such as “first”, “second”, “third”, etc. are arbitrarily assigned and are merely intended to differentiate between two or more instances or components. It is to be understood that the words “first”, “second”, “third”, etc. serve no other purpose and are not part of the name or description of the component, nor do they necessarily define a relative location, position, or order of the component. Furthermore, it is to be understood that the mere use of the term “first”, “second”, or “third” does not require that there be any “fourth” component, although that possibility is contemplated under the scope of the present disclosure.

For the purposes of describing and defining the present embodiments it is noted that the terms “substantially” and “approximately” are utilized herein to represent the inherent degree of uncertainty that may be attributed to any quantitative comparison, value, measurement, or other representation. The terms “substantially” and “approximately” are also utilized herein to represent the degree by which a quantitative representation may vary from a stated reference without resulting in a change in the basic function of the subject matter at issue.

According to a first aspect, a method of upgrading a feed stream may include introducing dilution steam to the feed stream, thereby forming a feed mixture; preheating the feed mixture; upgrading the feed mixture in an electrified hydrocarbon upgrading furnace to form a cracked gas effluent, wherein the electrified hydrocarbon upgrading furnace comprises one or more electrical heating elements for upgrading the feed mixture; introducing the cracked gas effluent to a first heat exchanger, thereby cooling the cracked gas effluent to a temperature in the range of 500° C. to 700° C.; and further treating the cracked gas effluent to form a cracked gas, wherein: a steam drum is fluidly connected to the first heat exchanger for transferring steam condensate to the first heat exchanger and saturated steam back to the steam drum, thereby forming a closed heat exchange loop, and the steam condensate transferred to the third heat exchanger is at a pressure in the range of 1 bar to 50 bar.

A second aspect may include the first aspect, and may further include wherein the feed stream further comprises hydrocarbon gases selected from the group consisting of ethane, propane, butane, or combinations thereof.

A third aspect may include the first aspect and may further include wherein the feed stream comprises hydrocarbon oils, refined hydrocarbons, bio-derived feeds, or combinations thereof, the hydrocarbon oils selected from the group consisting of naphthas, gas-oils, pyrolysis oils, or combinations thereof.

A fourth aspect may include any previous aspect, and may further include introducing the feed stream to a second heat exchanger prior to introducing the dilution steam to the feed stream, thereby preheating the feed stream to a temperature in the range of 23° C. to 263° C., and wherein the steam drum is also fluidly connected to the second heat exchanger for transferring steam to the second heat exchanger and steam condensate back to the steam drum.

A fifth aspect may include any previous aspect, and may further include preheating the feed mixture comprises introducing the feed mixture to a third heat exchanger, thereby preheating the feed mixture to a temperature in the range of 70° C. to 263° C.; and the steam drum is fluidly connected to the third heat exchanger for transferring steam to the third heat exchanger and steam condensate back to the steam drum.

A sixth aspect may include any previous aspect, and may further include preheating the feed mixture comprises introducing the feed mixture to a first e-heater; and the first e-heater preheats the feed mixture to from 250° C. to 400° C.

A seventh aspect may include any previous aspect, and may further include preheating the feed mixture comprises introducing the feed mixture to a fourth heat exchanger; the fourth heat exchanger is positioned downstream of the first heat exchanger on a cracked gas effluent side of the fourth heat exchanger; a temperature of the cracked gas effluent exiting the cracked gas effluent side of the fourth heat exchanger is less than a temperature of the cracked gas effluent exiting the first heat exchanger; the fourth heat exchanger preheats the feed mixture to a temperature in the range of 380° C. to 650° C. and cools the cracked gas effluent to a temperature of less than 700° C. to greater than or equal to 150° C.

An eighth aspect may include any previous aspect, and may further include preheating the feed mixture comprises introducing the feed mixture to a second e-heater, wherein the second e-heater is positioned immediately upstream of the electrified hydrocarbon upgrading furnace; and the second e-heater further preheats the feed mixture to a temperature in the range of 500° C. to 700° C.

A ninth aspect may include any previous aspect, and may further include wherein further treating the cracked gas effluent comprises exposing the cracked gas effluent to a quench fluid in a quench tower, thereby forming a quenched cracked gas mixture; and the quench fluid comprises water or at least one of diesel oil and gas oil.

A tenth aspect may include the ninth aspect, and may further include wherein further treating the cracked gas effluent further comprises introducing the quenched cracked gas mixture to a separator, thereby forming a recovered fluid and a cracked gas, the cracked gas comprising olefins.

An eleventh aspect may include any previous aspect, and may further include wherein the second and third heat exchangers further comprise one or more pumps interposed between the downstream end of the second and third heat exchangers and the steam drum.

A twelfth aspect may include any previous aspect, and may further include the method further comprises introducing excess steam from the steam drum to a third e-heater, thereby forming a superheated steam for export; and the second e-heater heats the steam from the steam drum to a temperature in the range of 120° C. to 500° C.

A thirteenth aspect may include any one of the first through eleventh aspects, and may further include introducing excess steam from the steam drum to a fifth heat exchanger, the fifth heat exchanger positioned downstream of the first heat exchanger on a cracked gas effluent side of the fifth heat exchanger; heating the excess steam from the steam drum to a temperature in the range of 120° C. to 500° C. in the fifth heat exchanger, thereby forming a superheated steam for export; and cooling the cracked gas effluent to a temperature of less than 700° C. to greater than or equal to 150° C. in the fifth heat exchanger, and wherein a temperature of the cracked gas effluent exiting the cracked gas effluent side of the fifth heat exchanger is less than a temperature of the cracked gas effluent exiting the first heat exchanger.

A fourteenth aspect may include any previous aspect, and may further include preheating the feed mixture comprises introducing the feed mixture to a sixth heat exchanger; the sixth heat exchanger is positioned downstream of the first heat exchanger on a cracked gas effluent side of the sixth heat exchanger; and the sixth heat exchanger preheats the feed mixture to a temperature in the range of 250° C. to 650° C., and cools the cracked gas effluent to a temperature of less than or equal to 350° C.

According to a fifteenth aspect, a method of upgrading a feed stream may include introducing the feed stream to a second heat exchanger, thereby preheating the feed stream to a temperature in the range of 100° C. to 263° C.; introducing dilution steam to the feed stream, thereby forming a feed mixture; further preheating the feed mixture; upgrading the feed mixture in an electrified hydrocarbon upgrading furnace to form a cracked gas effluent, wherein the electrified hydrocarbon upgrading furnace comprises one or more electrical heating elements for upgrading the feed mixture; introducing the cracked gas effluent to a first heat exchanger, thereby cooling the cracked gas effluent to a temperature in the range of 500° C. to 700° C.; and further treating the cracked gas effluent to form a cracked gas, wherein: a steam drum is fluidly connected to the second heat exchanger for transferring steam to the second heat exchanger and steam condensate back to the steam drum, as well as fluidly connected to the first heat exchanger for transferring steam condensate to the first heat exchanger and saturated steam back to the steam drum, thereby forming a closed heat exchange loop, and the steam condensate transferred to the third heat exchanger is at a pressure in the range of 1 bar to 50 bar.

Having described the subject matter of the present disclosure in detail and by reference to specific embodiments, it is noted that the various details disclosed in the present disclosure should not be taken to imply that these details relate to elements that are essential components of the various embodiments described in the present disclosure. Further, it will be apparent that modifications and variations are possible without departing from the scope of the present disclosure, including, but not limited to, embodiments defined in the appended claims.

1. A method of upgrading a feed stream, comprising:
introducing dilution steam to the feed stream, thereby forming a feed mixture;
preheating the feed mixture;
upgrading the feed mixture in an electrified hydrocarbon upgrading furnace to form a cracked gas effluent, wherein the electrified hydrocarbon upgrading furnace comprises one or more electrical heating elements for upgrading the feed mixture;
at least one of:
introducing the feed stream to a second heat exchanger prior to introducing the dilution steam to the feed stream, wherein a steam drum is fluidly connected to the second heat exchanger for transferring steam to the second heat exchanger and steam condensate back to the steam drum, or
introducing the feed mixture to a third heat exchanger, thereby preheating the feed mixture, wherein the steam drum is fluidly connected to the third heat exchanger for transferring the steam to the third heat exchanger and the steam condensate back to the steam drum; and
introducing the cracked gas effluent to a first heat exchanger, thereby cooling the cracked gas effluent to a temperature in the range of 500° C. to 700° C., wherein:
the steam drum is also fluidly connected to the first heat exchanger for transferring the steam condensate to the first heat exchanger and saturated steam back to the steam drum, thereby forming a closed heat exchange loop along with the second heat exchanger, the third heat exchanger, or both, and
the steam condensate transferred to the first heat exchanger is at a pressure in the range of 1 bar to 50 bar.

2. The method of claim 1, wherein the feed stream further comprises hydrocarbon gases selected from the group consisting of ethane, propane, butane, or combinations thereof.

3. The method of claim 1, wherein the feed stream comprises hydrocarbon oils, refined hydrocarbons, bio-derived feeds, circular feeds, or combinations thereof, the hydrocarbon oils selected from the group consisting of naphthas, gas-oils, pyrolysis oils, Fischer-Tropsch derivatives, natural gas condensates, or combinations thereof.

4. The method of claim 1, wherein introducing the feed stream to the second heat exchanger preheats the feed stream to a temperature in the range of 23° C. to 263° C.

5. The method of claim 1, wherein introducing the feed mixture to the third heat exchanger, preheats the feed mixture to a temperature in the range of 70° C. to 263° C.

6. The method of claim 1, further comprising introducing the feed mixture to a first e-heater the first e-heater preheats the feed mixture to from 250° C. to 400° C.

7. The method of claim 1, further comprising:
introducing the feed mixture to a third heat exchanger; and
introducing the feed mixture to a fourth heat exchanger, wherein:
the fourth heat exchanger is positioned downstream of the first heat exchanger on a cracked gas effluent side of the fourth heat exchanger,
a temperature of the cracked gas effluent exiting the cracked gas effluent side of the fourth heat exchanger is less than a temperature of the cracked gas effluent exiting the first heat exchanger, and
the fourth heat exchanger preheats the feed mixture to a temperature in the range of 380° C. to 650° C. and cools the cracked gas effluent to a temperature of less than 700° C. to greater than or equal to 150° C.

8. The method of claim 1, further comprising introducing the feed mixture to a second e-heater, wherein:
the second e-heater is positioned immediately upstream of the electrified hydrocarbon upgrading furnace; and
the second e-heater further preheats the feed mixture to a temperature in the range of 500° C. to 700° C.

9. The method of claim 1, further comprising exposing the cracked gas effluent to a quench fluid in a quench tower, thereby forming a quenched cracked gas mixture, wherein the quench fluid comprises:
water; or
diesel oil, gas oil, or both.

10. The method of claim 9, further comprising introducing the quenched cracked gas mixture to a separator, thereby forming a recovered fluid and a cracked gas comprising olefins.

11. The method of claim 1, wherein the second and third heat exchangers further comprise one or more pumps interposed between the downstream end of the second and third heat exchangers and the steam drum.

12. (canceled)

13. (canceled)

14. The method of claim 1, further comprising introducing the feed mixture to a sixth heat exchanger, wherein:
the sixth heat exchanger is positioned downstream of the first heat exchanger on a cracked gas effluent side of the sixth heat exchanger;
the sixth heat exchanger preheats the feed mixture to a temperature in the range of 250° C. to 650° C.; and
the sixth heat exchanger cools the cracked gas effluent to a temperature of less than or equal to 350° C.

15. A method of upgrading a feed stream, comprising:
introducing the feed stream to a second heat exchanger, thereby preheating the feed stream to a temperature in the range of 23° C. to 263° C.;
introducing dilution steam to the feed stream, thereby forming a feed mixture;
preheating the feed mixture;
upgrading the feed mixture in an electrified hydrocarbon upgrading furnace to form a cracked gas effluent, wherein the electrified hydrocarbon upgrading furnace comprises one or more electrical heating elements for upgrading the feed mixture;
introducing the cracked gas effluent to a first heat exchanger, thereby cooling the cracked gas effluent to a temperature in the range of 500° C. to 700° C.; and
further treating the cracked gas effluent to form a cracked gas, wherein:
a steam drum is fluidly connected to the second heat exchanger for transferring steam to the second heat exchanger and steam condensate back to the steam drum, as well as fluidly connected to the first heat exchanger for transferring steam condensate to the first heat exchanger and saturated steam back to the steam drum, thereby forming a closed heat exchange loop, and
the steam condensate transferred to the first heat exchanger is at a pressure in the range of 1 bar to 50 bar.
